(19) United States
(12) Patent Application Publication
(10) Pub. No.: US 2025/0195714 A1
(43) Pub. Date: Jun. 19, 2025

(54) DEVICES AND METHODS OF TREATING WOUNDS

(71) Applicant: University of Massachusetts, Boston, MA (US)

(72) Inventors: Raymond Dunn, Shrewsbury, MA (US); Jorge Rigel Lujan Hernandez, Worcester, MA (US)

(21) Appl. No.: 18/845,301

(22) PCT Filed: Mar. 10, 2023
(86) PCT No.: PCT/US2023/015010

§ 371 Date: Sep. 9, 2024

Related U.S. Application Data
(60) Provisional application No. 63/318,675, filed on Mar. 10, 2022.

(51) Int. Cl. A61L 15/44; A61K 31/196; A61L 15/64; A61L 26/00; A61L 27/54; A61L 27/58

(52) U.S. Cl. CPC A61L 15/44; A61K 31/196; A61L 15/64; A61L 26/0066; A61L 27/54; A61L 27/58; A61L 2300/418; A61L 2300/602; A61L 2300/622; A61L 2430/34

(57) ABSTRACT

Device and methods for treating wounds or surgical sites are provided. The devices and methods include use of antifibrinolytic agents such as tranexamic acid to reduce exudate and improve wound healing. Embodiments provide for the timed release of therapeutic agents from wound dressings to extend a delivery time through the inflammatory phase of wound healing.

This application claims priority to U.S. Provisional Application No. 63/318,675 filed on Mar. 10, 2022, the entire contents of this application being incorporated herein by reference.

The present disclosure relates generally to compositions and methods for treating wounds, including wounds that may produce significant amounts of exudates. The wound can include skin-graft donor sites, scald or other thermal injuries, surgical sites, or similar injuries or defects. The compositions and methods may also be used for internal sites, such as surgical sites in which open wounds are treated and closed.

Certain types of wounds, either due to surgery, trauma, burn, or other causes can present various challenges. For example, skin grafts and scald injuries result in loss of the epidermis and potentially a portion of the underlying dermis. The resulting wound sites can present challenges including production of large amounts of exudate. Typically, treatment includes repeated dressing changes and potential use of dressings or other devices (e.g., drains) adapted to absorb and/or remove exudate. The dressing changes can be extremely painful, and there can be difficulties in achieving rapid re-epithelialization. Although various new dressings and treatment methods have been developed over the years, there remains a need for improved methods for controlling wound drainage and promoting rapid wound healing.

Furthermore, some surgical procedures produce surgical sites that are more prone to complications from bleeding and/or exudate formation, including formation of seromas, which may require on-going drainage, and may result in delayed or less than optimal healing.

Accordingly, the present disclosure provides compositions and methods that can be used to improve treatment of various wounds including skin graft donor sites, scald injury sites, and other sites that have been affected by surgery, trauma, or other injury.

### SUMMARY

The present disclosure provides devices and methods for the treatment of wounds including a wound dressing for application of a therapeutic agent to a wound that reduces the generation of exudate from the wound and promotes wound healing. An exemplary embodiment provides for treatment of wounds in which a drain is not utilized as the amount of exudate is substantially reduced to a level that is less than the absorption capacity of the wound dressing. This enables a single application of the wound dressing to a wound site to complete the treatment of the wound. The wound dressing is applied with a therapeutically effective amount of the therapeutic agent distributed within the dressing for delivery at or through the tissue contact surface of the dressing. All or a portion of the dressing can comprise a biodegradable material depending on the type of wound being treated. The therapeutic agent can comprise a therapeutically effective amount of antifibrinolytic agent such as tranexamic acid (TXA) that serves both as a hemostat and is operative to reduce the generation of sanguineous and/or serous exudate from the wound. Depending on the type of wound, and the condition of the patient, other antifibrinolytic agents including epsilon-aminocaproic acid, aprotinin and alpha2-antiplasmin can be used to treat wounds to promote wound healing.

As antifibrinolytic agents are known to have side effects and a risk for allergic response there use has been restricted for topical treatment of trauma patients and for intravenous delivery for certain cardiac and arthroplasty procedures, for example. These agents are known to facilitate hemostasis but have been contraindicated for use after the normal period for hemostatic treatment, i.e. for less than three hours after initial application to treat traumatic injury or in conjunction with fibrin tissue sealants to stop hemorrhages during surgical procedures.

The wound dressing can comprise a fibrous material that incorporates the therapeutic agent on and/or adjacent to the tissue contact surface at a dosage level suitable to treat the type of wound. Different wounds can have different requirements depending on the level of hemostasis required to stop bleeding. An injury to the dermal layer of skin, such as by abrasion for example, disrupts a concentration of microvascular capillaries that can bleed upon initial injury, but typically such bleeding is quickly arrested by the application of pressure. Deeper injuries to the subcutaneous layer of skin, such as in deeper split thickness or full thickness skin graft donor sites, can damage larger arteries that can prolong bleeding in some patients. Consequently, the dressing can have an initial dose of the therapeutic agent to quickly stop bleeding and can incorporate a second dose that is released after bleeding has stopped to quench the production of exudate from the wound. In some injuries, such as scald injuries, no bleeding occurs so that the dosage is selected to limit the generation of exudate. Venous ulcers are a further class of wound where the dose may depend on the size and severity of the ulcer presented for treatment.

Preferred embodiments provide dressings configured to significantly reduce the production of exudate and thus enable treatment without the use of negative pressure or other drain devices. The therapeutic agents described herein have been demonstrated to quench the generation of exudate in normally highly exudative wounds so as to eliminate the need for changing the dressing during the inflammatory phase of wound healing process until re-epithelization of the skin, for example, in dermal wounds. Additionally, this treatment procedure did not extend the period of time needed for re-epithelization of the skin. For certain wounds that bleed more heavily, drains and/or negative pressure can be used but may also result in the removal of the therapeutic agent from the wound site so as to diminish effective treatment, or the amount of fluid exudate may saturate the wound dressing and possibly cause leakage from the dressing. In such cases, it can be advantageous to employ wound drainage for a limited time and configure the kinetics of delivery of the therapeutic agent to occur during periods in which drainage is not used.

The wound dressings described herein can utilize different delivery methods to control the delivery of the therapeutic agent. These can be used in conjunction with topical delivery for certain applications where an initial large dose can assist with hemostasis, however, a critical feature is to extend the delivery period beyond the period required for hemostasis into the inflammatory phase of the wound healing process. Thus, time release of the therapeutic agent is preferably used to limit the overall dose yet provide effective for the reduction in serous exudate during the inflammatory and proliferation phases, which can extend for days or weeks into the wound healing period.

Embodiments can employ porous sheets with particulates of the therapeutic agent distributed within the pores that release upon contact with fluid in the wound. Gels can also be used to regulate the delivery of medication into a wound. Further embodiments can utilize can microspheres in which the medication is retained until release. The dressing can also include an absorbent layer with sufficient absorbent capacity to retain exudate that is generated by the wound during the entire wound healing phase until healing is sufficient to remove the dressing without need for replacement. For skin graft donor site wounds for example, the period for single dressing use is typically at least 10 days after initial placement. The dressing can also include a fluid impermeable backing layer so as to prevent fluid leakage from the dressing. A peripheral adhesive layer can be used to attach the dressing to skin surrounding the wound in certain applications.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1A is a flow chart illustrating treatment options using the presently disclosed methods and compositions.

FIG. 1B schematically illustrates the wound healing process with the timeline showing different phases from the initial wound to complete healing.

FIG. 1C schematically illustrates different dose delivery options depending on the type of wound being treated as well as the size and severity of the wound.

FIGS. 2A and 2B illustrate steps for treating a skin graft donor site or similar wound according to exemplary embodiments.

FIG. 3 is a cross-sectional diagrammatic view of a wound treatment dressing according to exemplary embodiments.

FIG. 4 is a cross-sectional diagrammatic view of a wound treatment dressing according to other exemplary embodiments including variations in tranexamic acid (TXA) or other therapeutic antifibrinolytic medications distributed across a thickness of the dressing.

FIG. 5 is a cross-sectional diagrammatic view of a wound treatment dressing according to other exemplary embodiments including variations in TXA across a thickness of the dressing.

FIG. 6 is a cross-sectional diagrammatic view of a wound treatment dressing according to other exemplary embodiments including a controlled release method for TXA delivery.

FIG. 7 is a cross-sectional diagrammatic view of a wound treatment dressing according to other exemplary embodiments including a controlled release method for TXA delivery.

FIG. 8 illustrates an abdominal surgical incision in which a wound treatment device can be inserted for treatment of wounds or conditions.

FIG. 9 illustrates a biodegradable wound treatment device to be placed internally within a wound that provides for the treatment of the wound including the delivery of a therapeutic agent such as TXA to the wound.

FIG. 10 illustrates the optional use of a negative pressure wound therapy device in conjunction with the therapeutic treatments described herein.

FIG. 11 illustrates a cross-sectional view of an implanted wound therapy device that degrades over time and delivers therapeutic agents as described herein for the treatment of surgical wounds.

FIG. 12 illustrates a process sequence for treatment of wounds including the delivery of therapeutic agents described herein in combination with negative pressure wound therapy.

FIGS. 13A and 13B show top views of exemplary surgical implants as shown and described in connection with FIGS. 10-12.

FIG. 14 illustrates a wound treatment device for placement in a wound having optional channels for draining fluid from the wound.

FIGS. 15A-C illustrate wound closure devices used in conjunction with the delivery of therapeutic agents described herein.

FIG. 15D illustrates an exemplary dose delivery diagram in which an applied dose is sequentially delivered in combination with the application of negative pressure.

FIG. 16 illustrates use of a device for treatment of a breast including TXA.

FIG. 17 illustrates different sizes of particulate delivery for the delivery of one or more therapeutic agents to a wound to reduce to level of exudate during wound healing.

FIG. 18 schematically illustrates the use of particulate delivery distribution in a wound filler for placement in a flap wound or other internal wound site with release of medication from both sides.

### DETAILED DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purposes.

Devices, methods, and compositions for treating wounds have improved greatly over the last few decades. However, there remain challenges for treating certain wounds, including wounds prone to form large amounts of exudate or surgical sites prone to form seromas and/or hematomas. Such wounds include skin graft donor sites and scald injury sites.

The presently discussed devices, methods, and compositions can be used to treat a variety of wound sites. In general, the disclosed devices, methods, and compositions can be used to treat wounds prone to formation of exudate. Such wounds can include any site with removal of a portion of the epidermis, and optionally, part or all of the underlying dermis. It is also contemplated that wounds with deeper subdermal damage may be treated.

Wounds with epidermal damage or removal can result from a variety of injuries or surgical procedures. For example, epidermal injury and/or removal can results from scald, abrasion, other thermal injuries, radiation injury (e.g., severe sunburn), or chemical damage, including intentionally induced chemical injuries from chemical peels or similar cosmetic procedures. Furthermore, the aforementioned epidermal injuries can also result, if severe enough, in damage to dermal or subdermal structures. Likewise, surgical procedures, such as skin graft harvesting or removal of tissue for other reasons (e.g., cosmetic removal of skin discolorations) may be suitably treated using the disclosed methods, devices, and compositions.

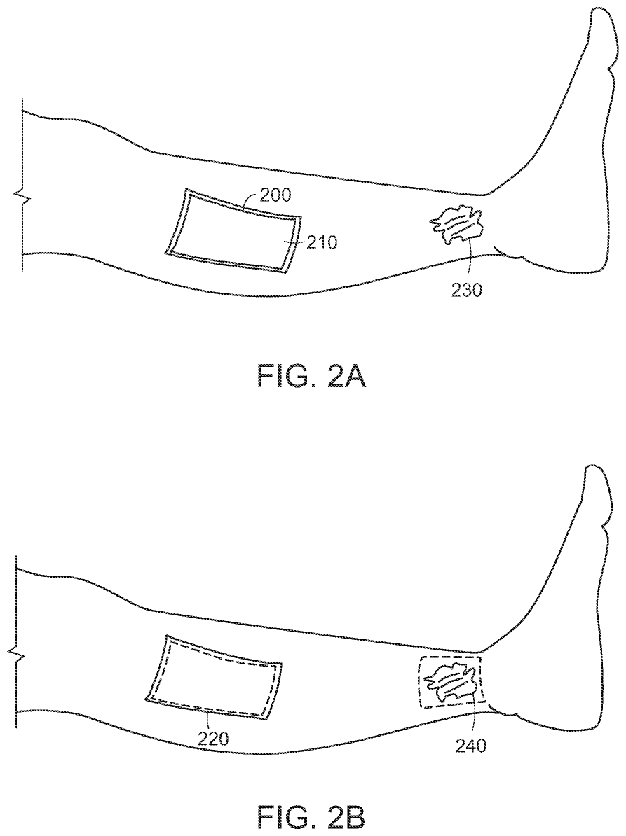

In addition, it is contemplated that the present methods, devices, and compositions may be used to treat various types of chronic wounds, especially those subject to exudate production. Such wounds may include, for example, ulcers (e.g., from bedsores), diabetic ulcers, venous stasis ulcers (illustrated as wound 230 in FIG. 2A, with dressing 240 in FIG. 2B), or difficult to treat traumatic injuries. Wounds resulting from surgical intervention including the treatment of surgical flap procedures can be treated with the devices and methods described herein wherein the use of drains to remove exudate can be substantially reduced or eliminated. In some embodiments in which drainage is used for a period of time, the period and/or the rate of therapeutic delivery can be adjusted to correlate with times in which drainage is not used or reduced so as not to substantially interfere with delivery. Devices and methods for the use of drainage from wounds and the application of negative pressure for wound treatment are further described in U.S. application Ser. No. 16/762,003 filed May 6, 2020 as a US national phase of International Application No, PCT/US2018/059812, filed on Nov. 8, 2018, which claims priority to U.S. Provisional Application No. 62/583,376 filed on Nov. 8, 2017, the entire contents of the above referenced applications being incorporated by reference herein in their entirety. The above incorporated applications describe the use of hydrogels for treatment of skin graft donor site wounds and other surgical wounds including dressings for the delivery of medications for treatment of wounds. The treatment of such wounds can also be combined with the use of drainage devices and the application of negative pressure to provide for the removal of exudate from the wound. Composite hydrogel formulations such as alginate hydrogels and membranes have been used been used to encapsulate TXA for use as a hemostatic agent. See Xie et al, Application of Alginate-Based Hydrogels in Hemostasis, Gels, 2022 February; 8 (2): 109, the entire contents of which is incorporated herein by reference. U.S. Pat. No. 10,314,950, the entire contents of which is incorporated herein by reference, describes the fabrication of alginate hydrogels that can be engineered to provide for the timed release of tranexamic acid, for example. This hydrogel comprises a crosslinked network of polymers that form a composite structure that can encapsulate antifibrinolytic agents for delivery to a wound.

In addition, as discussed further below, the presently described methods, devices, and compositions can be used for treatment of surgical sites, particularly those that may be prone to formation of seroma or excess formation of fluids. Such sites can include surgical sites that result in formation of voids or separation of tissue planes. For example, suitable sites may include areas where tissue has been removed or repositioned. Such sites can include areas where removal of fat or other tissue occurred (e.g., liposuction), movement of skin was needed, breast reconstruction or reduction, other breast procedures (e.g., mastopexy, augmentation, or reconstruction with use of expanders and/or implants), abdominoplasty, rhytidectomy for treatment of the face or neck, or other plastic or reconstructive procedures. In addition, the devices, methods, and compositions may be useful any place where tissue planes have been separated (e.g., after abdominal surgeries where different fascial layers are manipulated such as abdominoplasty). Further details describing devices and methods for the treatment of wounds for use in conjunction with the devices and methods described herein can be found in U.S. Pat. No. 9,597,484 that issued on Mar. 21, 2017, U.S. Pat. No. 9,226,737 that issued on Jan. 5, 2016, U.S. Pat. No. 9,301,742 that issued on Apr. 15, 2016, and U.S. Pat. No. 10,575,991, that issued on Mar. 3, 2020, the entire contents of each of the above referenced US patents and related applications being incorporated herein by reference. These devices and methods can be used for treatment of skin flap wounds, for example, wherein the wound dressing can be inserted into the wound for delivery of medications in which the dressing can comprise a biodegradable sheet having apertures extending through the sheet to enable tissue contact through the sheet of material to stabilize the wound during healing. The devices and methods can be used in combination with the use of drains and the application of negative pressure where needed to aid in the removal of exudate. The dressing can be further configured to deliver a therapeutic dose of an antifibrinolytic agent as described herein.

Managing wound exudate is important in promoting healing. In addition, large amounts of exudate can be uncomfortable for patients who may need multiple dressing changes, or may require drains and/or suction devices to remove and collect exudate from the wound site. Improved devices, methods, and compositions that greatly reduce exudate would, therefore, be beneficial. However, such devices, methods, and compositions should not interfere with necessary healing processes, including epithelization of skin graft donor sites or similar wounds.

One method for controlling bleeding includes application of tranexamic acid (TXA) to wounds. For example, Reyes et al. reported use of local TXA irrigation to decrease bleeding and reduce drainage for the first twenty-four hours after rhytidoplasty (face lift). See, Reyes, H M S et al. “Tranexamic acid: a simple way to reduce drainage and bleeding in rhytidoplasty,” Eur. J. Plast. Surg. (2021) 44:189-196. Furthermore, numerous others have reported use of TXA administered via a variety of routes for control of bleeding.

Despite the interest in use of TXA for control of bleeding, TXA has not been shown to control wound exudate for skin wounds such as scald injury or skin graft donor sites. Further, the limited research relating to topical application of TXA leaves unresolved concern about possible adverse effects of TXA. For example, Eikebrokk et al. studied use of TXA in keratinocyte and fibroblast cell cultures, and noted “Although short exposure to even high concentrations of topical TXA seems well tolerated in vitro, prolonged exposure can be cytotoxic and may affect wound re-epithelialization.” See, Eikebrokk, T A et al., “Cytotoxicity and effect on wound re-epithelialization after topical administration of tranexamic acid,” BJS Open 2019 Sep. 26; 3 (6): 840-851. Furthermore, other studies of TXA in various surgical contexts suggest a continued need for use of drains and do not provide devices or methods suitable to control exudate without ongoing use of drains and/or continual dressing changes.

The present inventors have discovered that topical application of TXA to skin graft donor sites and other wounds can produce a number of favorable results. Specifically, TXA was found, as discussed in describing in vivo treatment scenarios below, to greatly reduce exudate from skin graft donor sites. Further, the wounds showed rapid and complete or near complete re-epithelialization and little bleeding. Accordingly, the present inventors describe herein devices, methods, and compositions that have been shown in patients to be unexpectedly effective for treating skin graft donor sites, scald injuries, and other wounds or sites using TXA without complication.

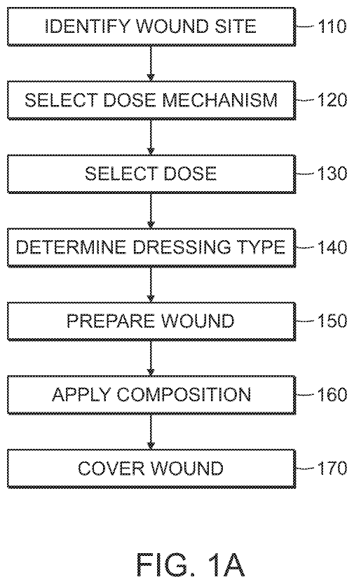

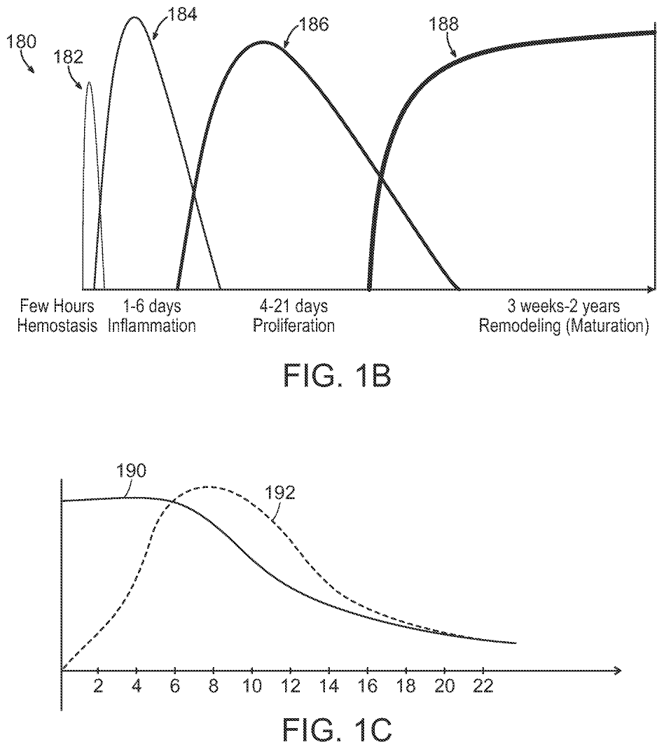

TXA is believed to competitively and reversibly inhibit the activation of plasminogen via binding at several distinct sites, including four or five low-affinity sites and one high-affinity site, the latter of which is involved in its binding to fibrin. The binding of plasminogen to fibrin induces fibrinolysis, and TXA prevents this dissolution of fibrin, thereby stabilizing clots and preventing bleeding. TXA and EACA both inhibit binding of plasmin to fibrin by occupying the lysine binding sites of the proenzyme plasminogen. This mechanism of action explains how TXA and other antifibrinolytics can reduce bleeding. However, the present inventors have surprisingly discovered that TXA can greatly reduce production of serous exudate. FIG. 1A describes the method of treatment described in further detail below, while FIG. 1B illustrates the phases of the normal wound healing process that extends form the time of injury 1002, through the inflammatory 1004 and proliferation 1006 phases until final wound healing after tissue has reformed at the wound such as by completion of re-epithelization.

Exudate at the donor site appears immediately after harvesting of the skin graft/creation of a partial thickness wound. The exudate is initially sanguineous in appearance during the first 24-48 hours but becomes serous over time. It has been reported that exudates up to 0.35 ml/cm² a day can be produced. Some research suggests that the exudate in partial thickness wounds after split thickness graft harvesting is a combination of early blood loss followed by inflammatory exudate as demonstrated by the increased number of inflammatory cells, proteins, immunoglobulins, and a decreased number of red blood cells in comparison to same patient's venous blood sample. This inflammatory nature was also noted and further investigated by others. Some researchers found that partial thickness wounds can generate exudate on average 0.45 ml per cm² a day and taper off to 0.25 ml per cm² up to three weeks after.

FIG. 1A is a flow chart illustrating treatment options using the presently disclosed methods and compositions. Generally, the process starts by identification of a wound or surgical site to be treated, Step 110. As discussed above, the wound site can include a variety of exudative or other wounds prone to produce exudate, bleed, and/or form seromas or hematomas. The wound may be identified in advance, e.g., in the case of a skin-graft donor site or other surgical procedure, or may be identified due to an unexpected injury such as a scald or other burn injury.

Next, an application/dose method (Step 120), dosing amount (Step 130), and dressing type (Step 140) are identified. In some cases, an initial dose of TXA may be applied as a topical solution, in an amount and distribution as described above. Alternatively, or additionally, a dressing containing TXA, for example, may be applied. In some cases, the dressing may contain no TXA but may be applied over the wound after application of a topical TXA solution. In other cases, the dressing may supply the TXA without topical application or may supply additional TXA. As discussed further below, the dressing can include variations in TXA amounts and can be configured to release TXA over time.

After selection of the application method (topical and/or dressing delivery), amount, and dressing type, the wound may be prepared as needed (Step 150). For example, often it will be necessary to clean the wound, remove certain tissues (e.g., residual epidermis), or treat adjacent structures. After proper preparation, TXA can be applied (Step 160) along with a dressing 210 (as shown in FIG. 2A), and the wound 200 can be covered (Step 170) with a sheet 220 to seal the wound and prevent exudate leakage (as shown in FIG. 2B). As discussed further below, the dressing may incorporate a backing sheet to allow application of the dressing and sealing/covering sheet in one step.

There are currently many types of dressings and implantable devices that can be used to treat wounds or surgical sites. Such devices can be configured primarily to manage exudate by absorption or wicking of fluids, but can also be configured to improve biologic processes, e.g., by preventing infection or improving conditions to allow growth of desirable tissue types or preventing formation of undesirable scar tissue. As discussed further below, TXA can be incorporated into or on various dressings or implantable materials to provide a desired amount of TXA soon after initiation of treatment and/or over an extended time (e.g., days or weeks) after wound formation. FIG. 1C shows examples of two different dosing regimes where the solid line indicates a dose level that is highest when applied at the time of injury, for example, as generally occurs in the treatment of skin graft donor sites where the dressing is applied immediately after removal of the skin graft. However, with other wounds such as scald wounds or venous ulcer wounds, there may be little or no therapeutic applied at the time of injury and there may be some gap until an effective dose can be applied during the inflammatory phase (dashed line). Preferably the antifibrinolytic agent is time released from the wound dressing for at least one day (24 hours) after the period of hemostasis of the wound, or more preferably for at least two days (48 hours) or more after the period of hemostasis. Note that for certain wounds such as burn or scald injuries, hemostatis occurs at the time of the injury and this is understood to be a situation where the hemostasis period is instantaneous and to have concluded prior to the application of the dressing to the wound.

In the simplest configuration, the dressing can include a flexible, relatively porous sheet of material that has a small amount or significant amount of absorptive capacity. For example, one suitable dressing type is TIELLE™ a product sold by 3M Corporation and described as a non-adhesive dressing that is a foam dressing designed for patients with low to highly exuding wounds. TIELLE™ generally includes a polyurethane foam layer that absorbs exudate. In addition, TIELLE™ includes a wicking layer between the polyurethane foam and an adhesive backing layer. The wicking layer can facilitate even distribution of exudate.

Although TIELLE™ is described herein as an exemplary dressing that may be applied over a wound treated topically with TXA, it should be appreciated that various modifications to the dressing or alternative dressings (e.g., including polyurethane or other materials) may be used. For example, the dressing can be made in a range of suitable thicknesses or sizes depending on the wound size and expected exudate. Further, the dressing may have a thinner or less absorbent material than used for typical exudative wounds with the expectation that less exudate will be produced with TXA use.

The dressing material can be selected based on a number of factors, including, for example, ability to absorb fluids (i.e., water-containing exudate). Typical dressings can absorb 0.25 ml to 2.5 ml per square cm of dressing, but it is contemplated that when using TXA, dressings with absorption capacity on the lower end may be acceptable.

Furthermore, other foam materials may be selected besides polyurethane. For example, exemplary foam materials can include alginate, silicone foams (e.g., 3M™ TEGADERM™ Silicone Foam Dressing) or fiber-based dressings such as carboxymethylcellulose (e.g., AQUACEL®, CONVATEC).

The dressing absorbent layer may be formed of a biocompatible material that does not degrade or become resorbed during the typical usage period. As such, the material will remain intact and can be removed after a specified time (e.g., after expected re-epithelialization for skin graft donor sites), or removed if needed to inspect the wound or replace a dressing.

In some cases, the dressing may include gelling fibers, such as the aforementioned carboxymethylcellulose, or hydrogels. Gelling fiber dressings form a gel when they come into contact with wound exudate, allowing them to effectively lock in excess fluid and at the same time provide enough moisture for healing to progress. Alginate, chitosan, polyvinyl alcohol and carboxymethylcellulose are examples of gelling fibers. Additionally, dressings are available that incorporate combinations of gelling and non-gelling materials. As such, dressings made of combinations of gelling and non-gelling materials are within the scope of the present invention.

TXA as used in the disclosed dressings may be incorporated into or attached to gelling or non-gelling materials. For example, in the case of a dressing that includes gelling materials, TXA may be bound to or embedded within the gelling material, for example, as a dry or semi-hydrated component. As the dressing comes in contact with fluid, either from exudate or application by a clinician, the TXA can be released from the gelling material to contact the wound. As such, the gelling material of the dressing can act as control on the amount of TXA delivered in relation to the amount of exudate being formed.

Alternatively, the TXA can be incorporated into and located in between one or more layers of the dressing. For example, some dressings include multiple layers, as discussed below, and the TXA can be positioned within one or more layers, or positioned between layers to hold the TXA in place until it comes in contact with fluids. Additionally, or alternatively, the TXA can be formed as a dressing layer, e.g., embedded in a water-miscible material or other substance that will allow the TXA to become biologically available when desired.

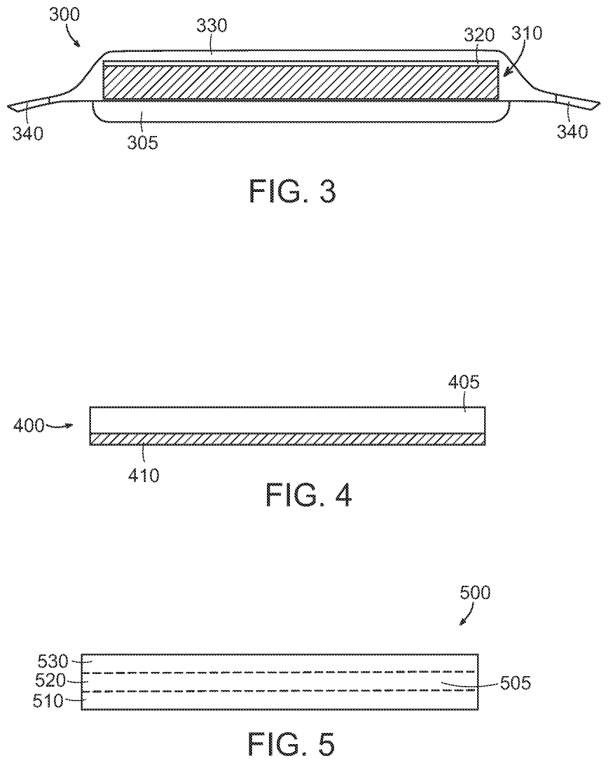

Exemplary dressings and other treatment devices are described below with reference to the following figures. FIG. 3 is a cross-sectional diagrammatic view of a wound treatment dressing 300 next to a wound 305 according to exemplary embodiments. As shown, the dressing includes a foam or other absorbent layer 310, an optional lateral wicking layer 320, and a backing layer 330. The edges of the backing layer may include an adhesive surface 340 that surrounds the wound and attaches to skin, thereby sealing the wound edges preventing or decreasing leakage and preventing wound contamination. Generally the backing layer 330 will be impermeable to liquids or pathogens from the outside but may be vapor permeable to allow some fluid evaporation from inside the dressing. In some cases, the dressing 300 includes only the absorbent layer 310, which may contain TXA, and additional layers such as a wicking layer or backing layer may be applied separately at a clinician's discretion.

The dressings described with respect to FIG. 3 or other figures can have a range of sizes depending on the site to be treated. For example, a suitable dressing may be between 5-500 cm². A typical skin graft donor site may be 50-300 cm² and as such, dressings may be provided in a range of sizes that allow coverage of wounds between these ranges, but it will be appreciated that larger and smaller sizes are contemplated.

Typical dressings can be rectangular, but other shapes may be desirable (e.g., for various anatomic locations). A square or rectangular dressing may have dimensions of 2 cm×2 cm, 2 cm×4 cm, 5 cm×5 cm, 5 cm×10 cm, 10 cm×10 cm, 10 cm×20 cm, 15 cm×20 cm, 20 cm×30 cm, or ranges in between. Other dressings may be ovoid, round, butterfly, triangular, or irregularly shaped to match various anatomic locations.

The absorbent layer 310 can contain TXA in various forms, concentrations, and/or distributions. For example, the TXA can be present in a solution, suspension, or dry form. In a solution or suspension, the TXA may be contained in a biocompatible solution or buffer such as a saline solution or degradable gel or hydrogel. If provided in dry form, the TXA may be distributed throughout the dressing or localized, e.g., concentrated near the wound contacting surface so that the TXA dissolves once contacted with body fluids or liquid such as saline applied to the wound.

The TXA can also be incorporated into the wicking layer 320, or between the wicking layer and backing layer. As such, as wound exudate is drawn into the dressing, the TXA will come in contact with fluid, thereby allowing the dry or otherwise provided TXA to dissolve.

In some cases, the TXA is present in the dressing with variations in TXA amount at different parts of the dressing. For example, FIGS. 4 and 5 are a cross-sectional diagrammatic views of wound treatment dressings according to other exemplary embodiments including variations in TXA across a thickness of the dressing. FIG. 4 illustrates a dressing 400 having an absorbent material 405 with TXA found in a wound contacting layer 410. As such, the TXA is attached to or embedded in the dressing 400 at an area close to the wound to allow rapid availability immediately after the dressing 400 is placed over a wound. The remainder of the dressing may be free of TXA initially or may contain smaller amounts of TXA per unit volume.

The TXA containing portion/wound contacting layer 410 can be formed of a material that is different than the absorbent material 405 (i.e., as a distinct material layer), or the same material. Further, the TXA can be attached to the dressing 400 as a dry material (e.g., powder) or can be contained within another material that may be dissolvable or partially soluble in water to allow TXA release when in contact with fluids.

Instead of having the TXA localized near the wound contacting portion of the dressing, the TXA may be found in a concentration that varies across the dressing. For example, FIG. 5 illustrates a dressing 500 formed of absorbent material 505 with layers 510, 520, 530 having differing amounts of TXA. The layers can each have discrete concentrations of TXA, or alternatively, a gradient of TXA (e.g., decreasing as one moves further from the wound site) may be used.

As shown, the dressing 500 has three layers. The layers can represent separate materials that are joined or layered in the wound, or can represent regions with differing TXA concentrations. Further, although three layers are shown, it is contemplated that more or fewer layers can be used. In addition, in some cases, the TXA can be arranged with one or more regions lacking TXA. For example, an inner layer 510 and outer layer 530 may contain TXA, while a middle area 520 may have no or less TXA, thereby allowing the TXA to be released in one or more phases as exudate reaches different portions of the dressing 500.

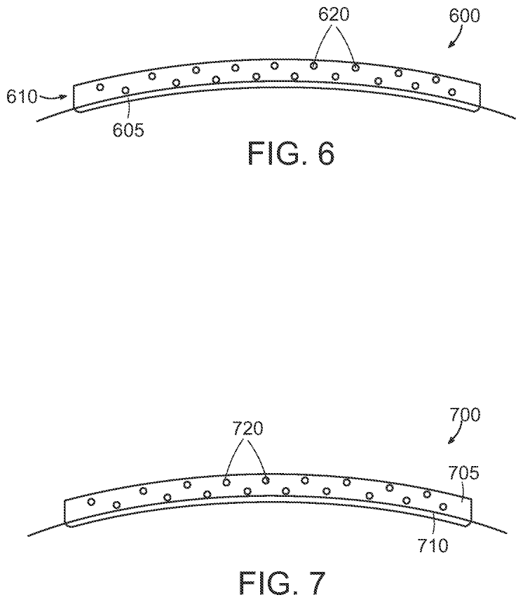

In some cases, in addition to or alternatively to controlling the concentration of TXA at different parts of the dressing, the TXA can be configured to provide for a controlled release or controlled biologic availability. FIG. 6 is a cross-sectional diagrammatic view of a wound treatment dressing 600 according to other exemplary embodiments including a controlled release method for TXA delivery. As depicted, the TXA is contained in or bound to materials that control the release of or biologic availability of the TXA, as discussed in more detail below. Generally, the TXA may be contained in or bound to a carrier material that can either degrade slowly in the presence of bodily fluids or allow release TXA over a predetermined period.

As depicted in FIG. 6, the dressing 600 has an absorbent material 610 and TXA particles 620. The TXA particles can include TXA contained in or attached to other materials that may dissolve at a desired rate when in contact with water, thereby making the TXA available to the wound 605.

Although the controlled release device of FIG. 6 is illustrated with TXA particles 620, it is contemplated that the TXA can be bound to or contained within materials having a variety of shapes and configurations. For example, the particles may be round, ovoid, oblong, or any other shape. In addition, the particles can take the form of fibers woven into or otherwise fabricated with (e.g., knitted, casted or extruded) the absorbent material. Further, the controlled release TXA may be contained in one or more discrete layers, similar to layer 320 of FIG. 3 or layer 410 of FIG. 4.

A number of materials may be selected to embed or encapsulate the TXA to allow controlled release. For example, in some cases, the TXA can be attached to or embedded in a gelling material such as a gelling-fiber material. Alternatively, the TXA can be contained in biocompatible polymers or other chemicals that will dissolve in contact with water. Such material may include, for example, gelatin, hyaluronic acid, or other materials that may be altered (e.g., by cross-linking) to control release rate.

In some cases, the TXA may be provided with a combination of an immediate release/initial dosage of TXA followed by a slow or controlled release to maintain a TXA concentration, which may be less than the initial dose. For example, FIG. 7 is a cross-sectional diagrammatic view of a wound treatment dressing 700 according to other exemplary embodiments including a controlled release method for TXA delivery. As shown, the dressing 700 includes an absorbent material 705 having a wound contacting layer 710 containing TXA and secondary TXA 720 distributed in the dressing 700. The TXA in the wound contacting layer 710 can be provided for immediate availability when the dressing is placed in contact with a wound. The amount of TXA in the layer 710 can be selected based on a desired immediate bolus at the start of treatment, and the secondary TXA can provide for a later or continuous supply over subsequent days. As such, the dressing can provide for immediate TXA delivery with a controlled amount of TXA as more exudate is produced and absorbed by the dressing. The therapeutic agent can also be partially or fully encapsulated in a biodegradable material such as a porous material in which the pores expand upon contact with fluids within the wound and thereby results in release of the therapeutic agent through the enlarged pores. Materials have specifically been fabricated for the controlled release of TXA and are described in U.S. Pat. No. 9,060,939 that granted on Jun. 23, 2015, the entire contents of which is incorporated herein by reference. TXA can thereby be encapsulated on or between layers or within cells of material that will provide timed release of TXA from a wound dressing.

As discussed above, the dressings can include at least an absorbent layer and backing or covering layer. It is contemplated that the dressings can be manufactured and packaged with the absorbing components and backing layer already assembled and ready to apply. However, it is also contemplated that a dressing having an absorbing material and TXA can be prepared and packaged without a backing layer or covering. As such, a surgeon or other user can apply the dressing along with the TXA and then apply a separate covering, which may be selected separately by the clinician or may be packaged as a kit with the dressing. As such, the surgeon or other user can select a backing material for a particular patient based on a number of factors such as wound size, patient specific characteristics (e.g., allergy to materials or adhesives), desired degree of backing flexibility or elasticity, vapor permeability, color, transparency, or other factors.

The backing or other material layers may be selected to provide a desired degree of strength (e.g., tear strength), flexibility, or elasticity. For example, a backing layer may be selected to have elastic properties similar to those of surrounding skin, thereby allowing the dressing to move with skin. The dressing may alternatively be stiffer, e.g., to prevent movement around the wound, or more elastic and flexible to allow expansion of the dressing if needed.

Various backing or covering materials that may be formed as an integral part of the dressing or applied over a dressing can be used. For example, suitable backing or covering materials can include 3M™'s TEGADERM™ or IOBAN™ materials. Other materials can include silicone sheets, polyurethane non-woven sheets, or other suitable materials. The backing may have an adhesive built in or, alternatively or additionally, a skin adhesive may be applied around but generally not in contact with the wound.

The dressings discussed above relate generally to dressing materials having a certain degree of absorbent capacity that are intended to be removed after a specified period. However, it should be appreciated that other materials can be selected, and those materials can include materials intended for later removal (e.g., after sufficient epithelization) or materials that are intended to stay in place permanently and may act as scaffolds to support tissue growth; control the type, direction, or rate of tissue growth; provide additional mechanical support to the implantation site; or control other biologic factors.

An exemplary alternative material that may be used for dressings or devices of the present disclosure include nanofiber materials. Such nanofibers can be made using a variety of processes such as electrospinning, and can include materials such as collagen, elastin, or polymeric materials such as polycaprolactone.

In some cases, the dressing or implantable device can include a scaffold material that can help support ingrowth of cells and formation of tissue without excessive fibrosis or scar formation. Such dressings or implants may be selected for implantation (e.g., to prevent seroma formation or for implantation to deliver TXA to specific tissues) or for treatment of skin where significant dermal or subcutaneous damage has occurred. Exemplary scaffold materials can include collagen-based materials, tissue matrices derived from tissues such as dermis, momentum, small-intestinal layers, pericardium, muscle, fat, or other tissues that can be processed to produce biocompatible scaffold. In addition, scaffold materials can be formed from biocompatible polymers such as polylactic acid or Poly(4-hydroxybutyrate) (TephaFLEX®). Alternatively, when used to treat skin wounds such as burns or severe scalds, the TXA may be used along with various burn treatment scaffolds such as INTEGRA® (INTEGRA LIFESCIENCES), which is a collagen-based dermal matrix template. Alternatively, the scaffold material can include one or more acellular tissue matrix or synthetic materials that provide mechanical support and/or allow tissue ingrowth. For example, suitable tissue matrices can include acellular dermal materials (e.g., ALLODERM® or STRATTICE®, both sold by LIFECELL CORPORATION®), or other acellular tissues such as small intestine submucosa, acellular bladder layer(s), or other materials used for surgical and regenerative medicine. Suitable synthetics can include VICRYL® (polyglactin 910) or other synthetics.

When used for internal sites, the scaffold can be left in place to provide mechanical support, improve tissue regeneration, and deliver TXA. As discussed above, such scaffold materials can be implanted at any site where control of exudate formation (e.g., to prevent seroma) is desired. Such sites can include any site where tissue has been removed or tissue layers have been separated to form a void where fluid may accumulate. In addition or alternatively, the sites can include areas where an implant, tissue expander, or other foreign body may be implanted. For example, as discussed below, TXA may be provided along with a tissue matrix or synthetic at a breast treatment site that contains or may be later modified to contain an implant or tissue expander.

Also within the scope and claims of the present disclosure are methods of treatment using the disclosed methods and devices. FIG. 1 outlines various steps for performing these methods. In some cases, the method includes selecting a skin wound prone to formation of moderate to high amounts of exudate, applying TXA to the wound, and covering the wound with a dressing. The TXA may be applied as a topical solution and/or contained within a dressing (or both), as described previously. In some cases, the wound is covered with the dressing and/or sealing sheet, and the wound is allowed to heal until a desired time lapses or a desired amount of reepithelization or other tissue growth has occurred.

It is contemplated that the methods can be performed without use of drains, as the amount of exudate will be low. It is also contemplated that the methods can be performed and allow reepithelization in the case of skin wounds without subsequent dressing changes. The treatment time can vary based on the size, depth, and other factors relating to the wound, but may be from 3-10 days, 3-5 days, 7-14 days, or other specific intervals.

Additional therapeutic agents may also be incorporated into or added to the dressings or devices discussed herein. Such agents can be used with any of the dressing or device configurations discussed above. In particular, in some cases, it may be desirable to incorporate agents that inhibit growth of or kill pathogens such as bacteria, and/or improve re-epithelialization or other desired processes (e.g., prevent scar).

In some cases, a bacteriocidal or bacteriostatic agent may be included in the dressings or implantable devices. Suitable agents can include silver (as a salt or other form), antibiotics (e.g., sulphamylon), or other antibacterial or anti-fungal agents. The additional agents can be incorporated in a manner similar to the TXA described above, or can be applied in a separate form (e.g., a separate dried powder).

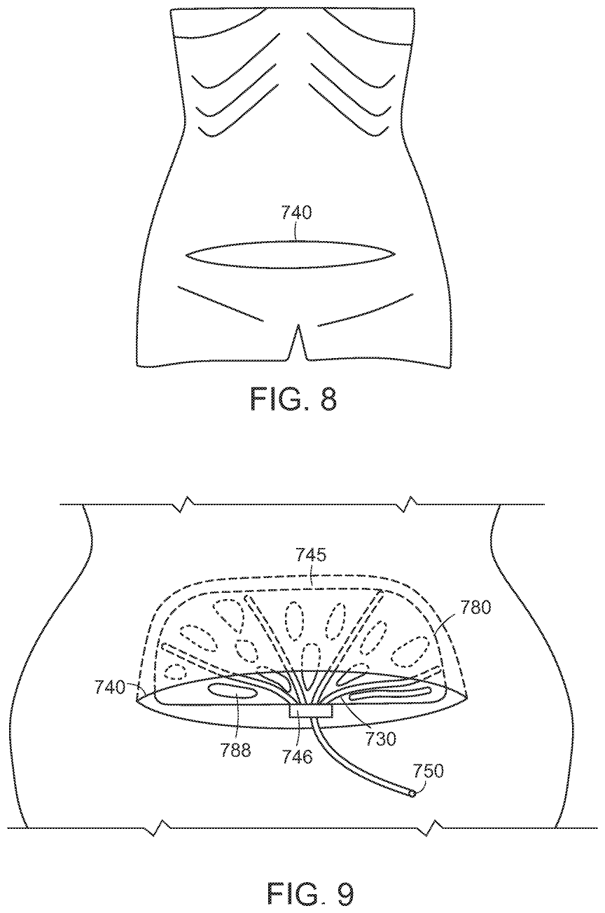

For the treatment of surgical wounds such as surgical flaps, a biodegradable matrix can be placed into the wound. Various biodegradable materials are discussed above, and such materials can include synthetics, acellular tissue matrices (e.g., ALLODERM®), or resorbable biologic materials (e.g., collagen-based scaffolds). FIG. 8 illustrates a surgical incision 740 in the abdomen of a patient; however, there are numerous other surgically treated conditions in which the devices and methods described herein can be used for treatment. FIG. 9 illustrates a surgical flap 745 in the abdominal area in which a device 780 has been inserted into the wound for treatment. The device 780 can comprise a layer of material that is preferably formed with spaced apart apertures 788 extending through the layer so as to provide for tissue contact through the apertures. In exemplary embodiments the device 780 can comprise a matrix with one layer of material or a plurality of layers having a wound conforming shape that is sufficiently thin and with apertures of a suitable size and shape so as to enable tissue located on opposite sides of the matrix to extend into the apertures so as to contact each other and thereby facilitate wound healing through the apertures. An amount of TXA is distributed within the one or more layers during fabrication or prior to implantation of the device into the wound site. The tissue contact surfaces on both sides of the matrix can be coated with an initial dose of the TXA for immediate release into the wound, one or more layers of TXA, or a distribution of TXA in particulate form that can provide for controlled release. In some embodiments the matrix is placed without the use of drainage as the TXA dosage is sufficient to treat the wound as the amount of exudate or seroma is reduced so that any exudate or seroma is re-adsorbed by the body.

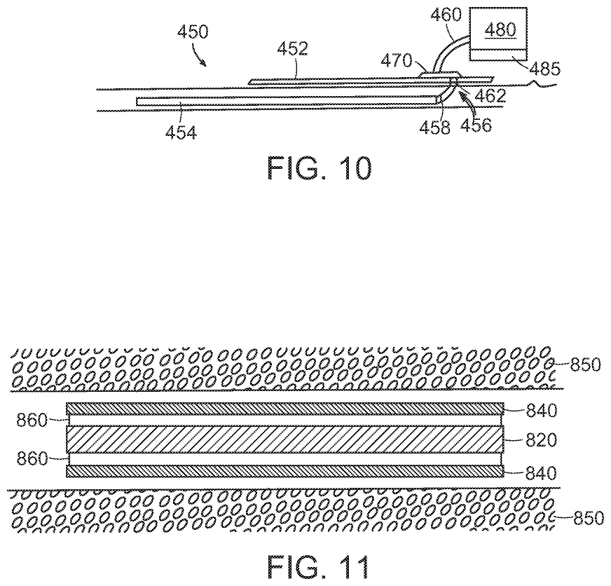

In certain procedures, the amount of seroma and/or exudate generated by the wound can exceed the capacity of the treatment device or dressing to absorb the fluid so as to necessitate the use of drainage. In such cases, one or more drain tubes can be used to provide drainage from the wound. In the example of the implant shown in FIG. 9, one or more drain tubes 730 can be inserted with the matrix 780 into the wound cavity 745. A manifold 746 can be used for draining a plurality of tubes through a single entry point thereby allowing the remainder of the wound opening 740 to be closed during a subsequent drainage period. The single tube or manifold 746 can be coupled to a single tube 750 that is fluidly coupled to a drainage reservoir or optionally to a negative pressure source such as a hand actuated or electrically operated pump. Shown in FIG. 10 is a cross-sectional view showing a system 450 including placement of a device 454 within the wound in which one or more tubes 458 extend to an external manifold 470 at tube coupling 462 wherein the one or more tubes 458 exit the wound at the single entry point 456. The wound is sealed with external drape 452 to provide a fluid impermeable barrier and an external tube is coupled to the negative pressure source 480 and collection reservoir. The source 480 or pump can be electronically controlled with controller 485 so as to be periodically operated in a programmed sequence. Additionally, one or more sensors can be used to measure fluid pressure within the wound to regulate application of pressure or the delivery of one or more therapeutic agents as described herein into the wound.

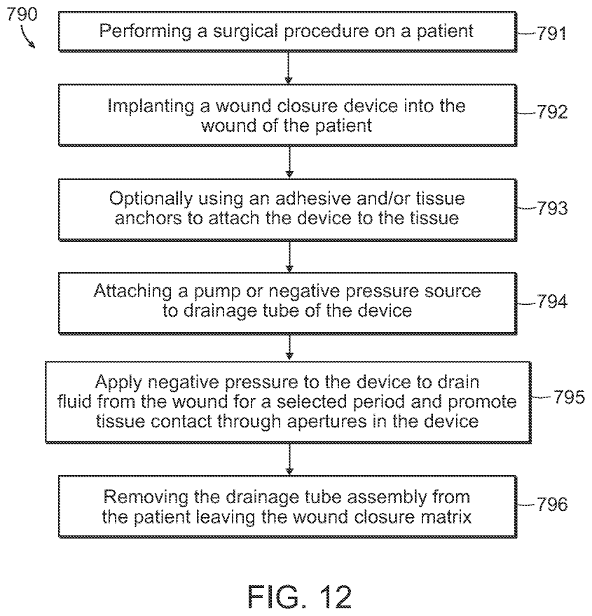

Shown in FIG. 11 is a cross-sectional view of an exemplary embodiment of a composite layered structure in which tissue 850 is located on opposite sides of the layered structure that can include apertures extending through the structure as described further in connection with FIGS. 12-14. The opposite sides of the structure can include layers of a therapeutic agent 840 that release TXA into the wound upon contact with the tissue. For example, initial layers 840 of TXA on opposite sides can be released initially onto the tissue surfaces within the wound cavity. The layers 840 can include a surface encapsulation layer that quickly degrades to release layers 840. The layers 840 will be released to expose biodegradable layers 860 having a selected thickness to degrade over a selected time period. This time period can be selected to include a drainage period, for example. After a selected time period, the central layer 820 can include a further dosage of TXA to be delivered into the wound after withdrawal of the drain tubes, for example.

Shown in the process flow diagram of FIG. 12 shows a treatment process 790 illustrating the steps in which after a surgical procedure is performed 791, a wound treatment device is implanted into the wound 792. Note that a medical adhesive can be used to secure the device 793 in position or close all or a portion of the wound in conjunction with suturing that may be needed. Tissue anchors can optionally be used on the tissue contact surfaces of the device to stabilize placement within the wound. If the option of negative pressure is to be employed to assist with drainage or wound closure, a pump is attached to one or more drain tubes 794. Negative pressure is then periodically applied 795, preferably during selected periods so as to limit or prevent the removal of any therapeutic compositions delivered into the wound with the device or local delivery. After any drainage, the tubes are removed 796 to leave the implanted device in the wound after closure.

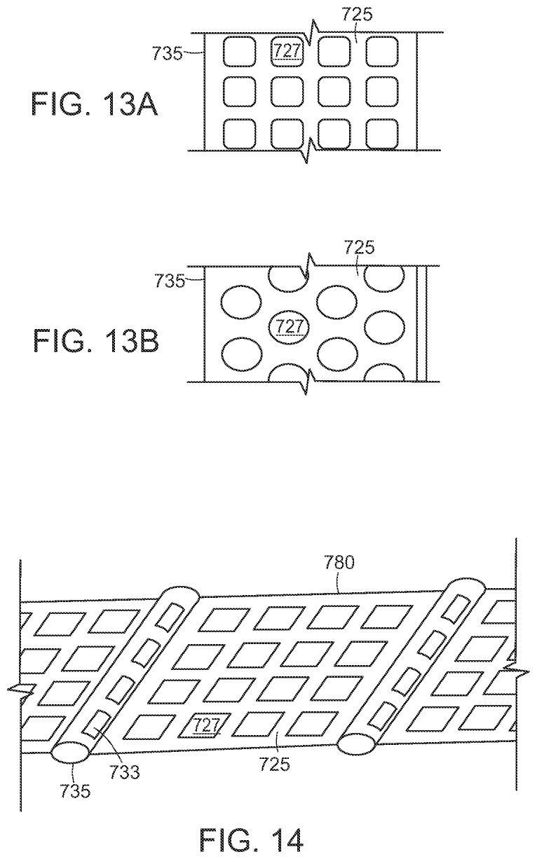

As shown in FIGS. 13A and 13B, the implanted device can include apertures 727 that extend through matrix or layered structure. The edges 735 of the structure can have a selected thickness in a range of 1-10 mm, for example. The top and bottom surface areas 725 between the apertures can have one or more layers of a therapeutic agent distributed thereon or within an interior layer or pattern of cells for controlled delivery over time. The apertures can be circular, oval, rectangular or other shape to facilitate tissue contact through the matrix. The matrix can also have a selected pore size and density to facilitate tissue ingrowth and time release of an internally distributed therapeutic agent such as TXA. FIG. 14 shows a further embodiment in which channels 735 can be included in the implant 780. The channels 735 can be sized to receive drain tubes as described previously herein so as to stabilize their position within the wound. This serves to position the tubes so as to leave the apertures open for tissue contact through the apertures, wherein the implant further has tissue contact areas 725 between the apertures so that the therapeutic agent embedded on or within the implant releases onto the surrounding tissue at a selected rate. The channels 735 can also have openings 733 to allow flow of fluid into the tubes. Alternatively, the channels can be sized and extend along the surfaces of the matrix to transport fluid to an external drain. The channels serve to transport fluid for a time period until the matrix degrades within the wound.

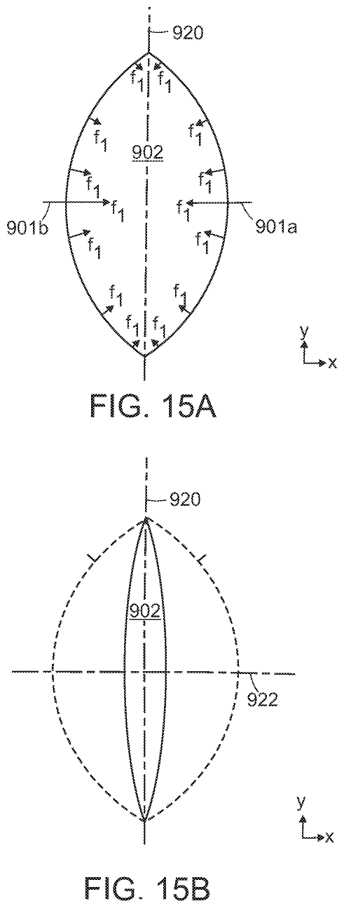

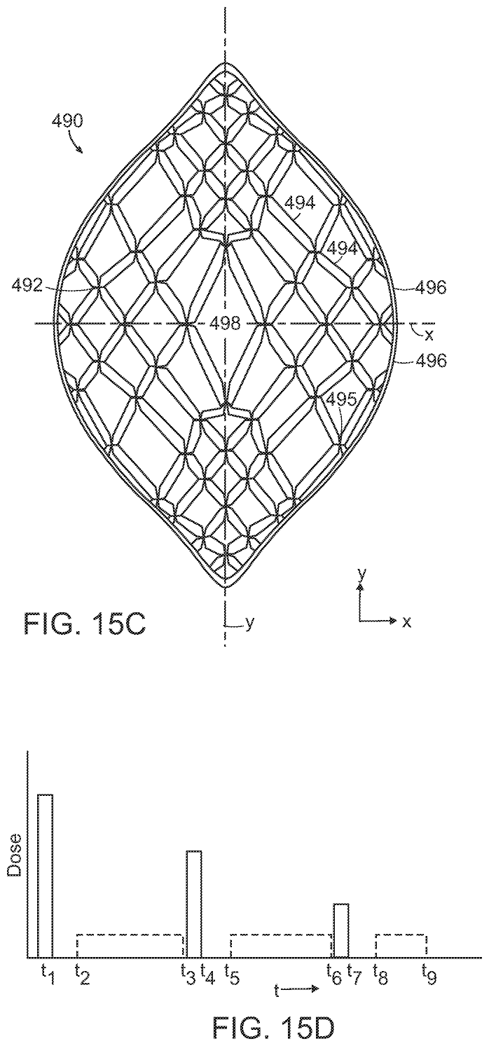

In further embodiments, the therapeutic agent, such as TXA, can be delivered with a wound closure device placed within and/or above the wound that facilitates closure of a wound along with the application of negative pressure. FIGS. 15A-15C illustrate various aspects of preferred embodiments of such a device. Many surgical wounds are formed with a linear incision through the skin where the edges separate so as to form a wound opening having an oval or elliptical shape. A wound closure device 902 having an oval shape with longitudinal and transverse axes 920, 922, respectively, can then be inserted into the wound as shown in FIGS. 15A and 15B. The device can be sealed in the wound with a drape as described previously, with a port extending through the drape that can be connected to a negative pressure source. The wound closure device can be configured to preferentially collapse along the transverse axis 922 as seen in FIG. 15B while inhibiting collapse along the longitudinal axis 920. The original size of the wound is depicted with the dashed line in FIG. 15B, however, due to the applied force distribution shown in FIG. 15A, the wound edges move towards the original line of incision. The applied force along the transverse axis moves in opposing directions 901a and 901b so as to cause movement of the wound margins towards closure of the wound over a selected period of time. The device thus moves from an initial open position seen in FIG. 15A to the more closed position in FIG. 15B with the margins of the wound generally maintaining contact with the peripheral outer wall of the device. An exemplary wound closure device 490 is shown in the top view of FIG. 15C in which a stabilizing structure comprises a plurality of cells 498 that extend at least partially between the top and bottom surfaces. The cells 498 can be open at the top and/or bottom in selected embodiments. The stabilizing structure can have cells of varying size and shape so as to facilitate lateral closure. The cells can have a diamond shape, a rectangular shape, an oval shape, a circular shape or various combinations thereof. The cells can be bounded by one or more walls 494. The walls can be connected by joints 492, 495 that can have different levels of flex so as to facilitate lateral collapse along the x axis and inhibit collapse along the y axis. The outer wall surfaces 496 can be symmetric around both axes. The stabilizing structure can comprise a foam, a felted foam or a composite structure of silicone and foam materials in which a therapeutic agent is distributed within the stabilizing structure for release into the wound. As the therapeutic agent, such as TXA, quenches the production of seroma, the wound closure device is configured to facilitate closure of the wound. The wound closure device can comprise a plurality of cells that collapse preferentially in one direction such that the margins of the wound move towards wound closure as described in the issued US patents previously incorporated by reference herein. The therapeutic agent can be delivered as a particulate where particles are integrated into the wound closure device as described herein and/or by fluid delivery from a source that can optionally be metered at a rate for delivery during periods in which negative pressure is reduced or paused to facilitate delivery onto tissue surfaces within the wound. This can substantially reduce the occurrence of seromas and infections during treatment. An exemplary diagram of a dosage delivery sequence is shown in FIG. 15D wherein the vertical bars indicate a first dose amount delivered for an initial time period t₁, followed by a time period t₁-t₂ in which the therapeutic agent is absorbed into tissue. At time t₂, negative pressure can optionally be applied for a time period indicated by dashed lines until t₃ by manual adjustment and control or by automated operation of the control system as described in connection with FIG. 10. At time t₃ the applied negative pressure is reduced or switched off and a second dose can be delivered to the wound until time t₄. Depending upon the circumstances of the patient and the type and condition of the wound, a second period of negative pressure can optionally be applied to the wound at time t₅ until time t₆. A third dose can then be delivered until time t₇ wherein the dose is absorbed into the tissue. Depending on the amount of exudate still being generated by the wound, an additional period t₈-t₉ (or periods) of negative pressure can optionally be applied, and or further doses of the one or more therapeutic agents described herein can be delivered to the wound. Note that one or more sensors can be used to detect the presence of a therapeutic agent in the wound at a selected concentration either using a chemical sensor that senses the compound of interest or indirectly such as by measurement of pH, or some characteristic indicative of the presence of the therapeutic agent of interest in the wound. Sensors can be placed into the wound with a dressing (at 458, at port 470, or within tube 460, for example) or wound therapy device as described herein or within a reservoir collection container to automate the application of negative pressure for selected periods. Alternatively, a sample of blood can be taken from the patient or a fluid sample from the wound can be analyzed by standard testing to determine concentration levels of selected constituents, such as indicators of inflammatory response (C-reactive protein (CRP), erythrocyte sedimentation rate (ESR) and procalcitronin (PCT)), to determine the stage of healing of the wound and whether further therapeutic treatment is required. In the event of infection of the wound, this indicates the need for delivery of an antibiotic, for example, into the wound in conjunction with TXA, for example.

Details regarding the use of a drainage system are further described in U.S. Pat. No. 10,166,148, issued Jan. 1, 2019, the entire contents of this patent being incorporated herein by reference. The drainage system can be turned on and off selectively during treatment so as not to remove the TXA or other medication(s) from the wound during a release period thereof into the wound. Thus, for example, the wound can be drained prior to release of the TXA and then negative pressure can be reduced or turned off during the release period, or the drain can simply be removed from the wound prior to release. The drainage system can selectively include the application of negative pressure to the wound using a negative pressure source connected to the dressing with a tube at a port through a fluid impermeable layer that is adherent to the skin of the patient around the wound.

As discussed above, the devices and methods can be used in various surgical procedures for treatment of breast, including procedures that employ acellular tissue matrices or synthetic matrices with or without an implant or tissue expander. For example, breast reconstruction or augmentation often includes use of a tissue matrix such as ALLODERM® or use of a VICRYL® mesh. The tissue matrix or synthetic can provide mechanical support, but depending on the material can also serve other important purposes. For example, acellular dermal materials can facilitate tissue ingrowth and regeneration, support vascularization, and reduce capsule formation or capsular contracture. However, surgical procedures of the breast or other sites (e.g., abdominoplasty sites) can be prone to seroma formation, which increases infection risk and may require extended drainage or, in a worst case, removal of an implant or expander due to infection. Accordingly, it is contemplated that TXA applied in or along with an acellular matrix, synthetic matrix, or implant can greatly improve breast surgery or other surgical outcomes.

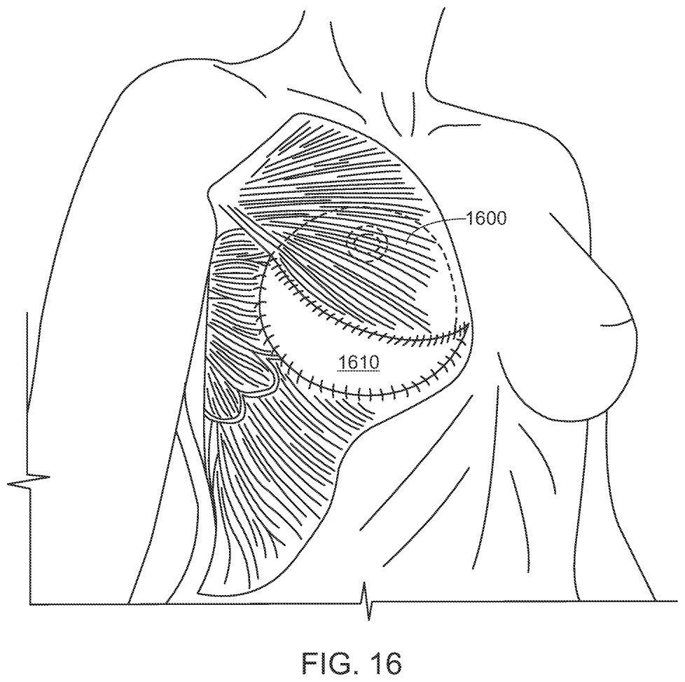

FIG. 16 illustrates an exemplary breast treatment site including an implant 1600 and matrix materials 1610. As shown, the implant 1600 is partially surrounded by the matrix 1610 such as ALLODERM®, but it is contemplated that the matrix can include a synthetic or other suitable material. The matrix can contain TXA in configurations discussed above.

It is noted that the matrix 1610 can be positioned as shown, but it will be appreciated that the location and type of implant may vary based on the specific procedure. For example, the matrix may be used in a reconstructive procedure to close the surgical site without an implant or expander, in a staged reconstruction, in a subpectoral or prepectoral procedure, or other variations on breast surgical procedures.

Accordingly, methods of treating a breast are also within the scope of the invention. The methods can include performing a breast procedure (e.g., mastectomy), implanting a material containing TXA, and closing the breast. The methods can also include implanting an implant or expander near the material containing TXA. The procedure may be performed with or without a drain.

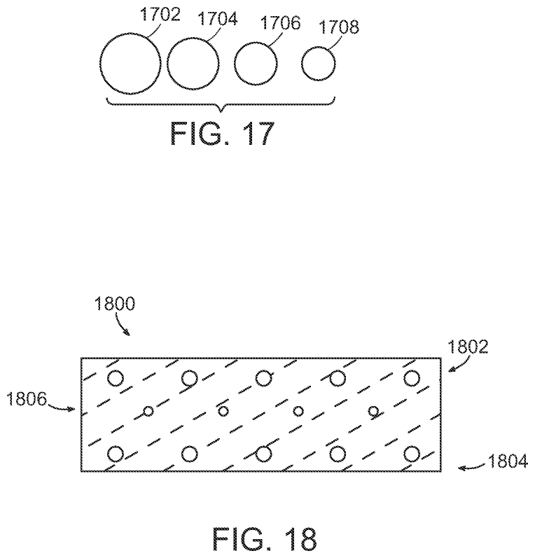

Shown in FIG. 17 are microspheres fabricated by methods to provide for the timed release of antifibrinolytic agents such as TXA or EACA from a wound dressing in which the microspheres are delivered. The microspheres can have a range of sizes 1792, 1704, 1706, and 1708 to retain different dosages to be released at different times upon application of the wound dressing to the patient. Examples of microspheres fabricated for release of TXA are described in Denry et al, Tranexamic acid loaded hemostatic nanoclay microsphere frameworks; J Biomed Mater Res B Appl Biomater, 2022 February; 110 (2): 422-430, the entire contents of which is incorporated herein by reference. Thus, nanoparticles fabricated as microspheres or discs approximately 25 nm in diameter from Laponite can have a porous structure with pore sizes in the range 3-6 microns that are suitable to deliver a time released dose of TXA from a wound dressing. Further methods for fabricating microspheres suitable for delivery of antifibrinolytic agents are described in U.S. Pat. Nos. 5,631,021 and 10,154,976, the entire contents of these patents being incorporated herein by reference. Tranexamic acid and salts thereof, such as Ca-TXA, can be delivered from polylactic-co-glycolic acid (PLGA) microspheres that biodegrade over a selectable period to provide controlled time release into a wound from a wound dressing in which they are dispersed. FIG. 18 illustrates a cross-sectional view of a wound dressing 1800, wherein opposite sides have microspheres or capsules 1802 and 1804 of a larger size for initial release from both sides, and a central layer of a smaller different size of microspheres distributed for later release from the wound dressing. The wound dressing 1800 can also be configured to deliver from a single side such as a dressing applied over a wound such as a skin graft donor site, scald or burn wound or a venous stasis ulcer wound as described previously herein.

### Example

Human patients were treated with TXA embedded dressings. A 50 cm² partial thickness wound was generated with a dermatome, thereby creating the skin-graft donor site. To prepare the dressing, one to two grams of TXA was dissolved in 10 ml normal saline. Non-adherent sterile dressings (TIELLE™) were contacted with the solution and placed directly on the donor site dressing.

The wounds were covered with a semipermeable film (TEGADERM™) without a drain. Normally a dermal drain is placed through the film into contact with the wound with the intention of collecting fluid. A total of 300 ml in 5 days is normally collected (60 ml per day=1.2 ml/m²). Thus, these patients were treated without the use of a drain where the original dressing was maintained for 12-14 days and then removed to expose a re-epithelialized skin surface without the need for placement of a further dressing.

These results suggest that TXA application topically can contribute to reducing significantly the amount of exudate to the extent of not requiring a fluid drainage system, but rather an absorbent material for a smaller amount of sanguineous and/or serous exudate. Importantly, the dressing was removed 8-16 days after application. In most cases the wound was fully epithelialized, with no adverse effect of TXA.

Similar dressings were used to treat scald injuries and chronic ulcers with similar reduction in exudate and near complete reepithelization. In particular, a child with a scald was treated with TIELLE™ containing TXA and the dressing was removed after ten days. Little exudate was noted and the wound was fully epithelialized.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

1. A device for treating a wound or surgical site, comprising:
a dressing material including a flexible sheet; and
a therapeutic amount of an antifibrinolytic agent for release from the flexible sheet to treat a wound having a period of hemostasis wherein the antifibrinolytic agent is released from the flexible sheet for a therapeutic period longer than the period of hemostasis to reduce the generation of exudate by the wound.

2. The device of claim 1, wherein the antifibrinolytic agent comprises tranexamic acid or a salt thereof, or aminocaproic acid or a salt thereof.

3. The device of claim 1, wherein the dressing material comprises a biocompatible polymer.

4. The device of claim 3, wherein the polymer comprises a foam, a woven material, a non-woven fabric, a knitted material, or combinations thereof and wherein the dressing material comprises two or more layers.

5. (canceled)

6. The device of claim 1, wherein the dressing material comprises at least one absorbent material layer or comprises at least one wicking layer and wherein the wound dressing is sized and shaped to treat a wound on a region of skin of a patient.

7-8. (canceled)

9. The device of claim 2, wherein the tranexamic acid (TXA) TXA has a concentration that varies across a thickness of the flexible sheet or the concentration of TXA varies between layers.

10. (canceled)

11. The device of claim 2, wherein tranexamic acid (TXA) TXA is distributed through at least a portion of the dressing material.

12. The device of claim 11, wherein the device comprises two or more layers, and TXA is enclosed between two layers.

13. The device of claim 1, further comprising a backing layer with an adhesive border that forms a fluid impermeable wound dressing for attachment to a region of skin around the wound.

14. The device of claim 1 further comprising an antimicrobial agent in the device or applied to the wound wherein the agent comprises a silver material or an antibiotic.

15. (canceled)

16. The device of claim 1 wherein the antifibrinolytic agent comprises epsilon-aminocaproic acid or wherein the antifibrinolytic agent is within a plurality of microspheres or porous particles distributed within the dressing or wherein the antifibrinolytic agent is dispersed within a hydrogel dressing material or an alginate hydrogel dressing material or wherein the antifibrinolytic agent is released upon contact with fluid from the wound for a time period of at least three hours after application to the wound or wherein the antifibrinolytic agent is released from the wound dressing after hemostasis and during an inflammatory phase of the wound.

17-20. (canceled)

21. The device of claim 1 wherein the antifibrinolytic agent is released for a time period including the hemostasis period and through a period of an inflammatory phase of the wound to reduce the generation of sanguineous and/or serous exudate from the wound or wherein the wound dressing provides for time release of the antifibrinolytic agent for at least one day (24 hours) or at least two days (48 hours) after the period of hemostasis of the wound or wherein the antifibrinolytic agent comprises tranexamic acid that is distributed within the wound dressing that includes a fluid impervious barrier that adheres to skin tissue around at least a portion of the wound.

22-23. (canceled)

24. The device of claim 1 wherein the wound dressing is configured to be placed over a skin graft donor site wound, a scald wound or a venous stasis ulcer wound.

25. A wound dressing for treating a dermal wound, comprising:
a dressing material including a flexible sheet comprising an adhesive, the flexible sheet configured to provide a fluid impermeable seal around the dermal wound; and
a therapeutic amount of an antifibrinolytic agent for release from the flexible sheet to reduce the generation of exudate by the dermal wound, wherein the antifibrinolytic agent is released from the flexible sheet for a therapeutic period longer than a period of hemostasis to reduce the generation of exudate by the wound.

26. The wound dressing of claim 25, wherein the dermal wound is a donor site of a skin graft donor site, the dermal wound being a scald wound or an ulcer.

27-29. (canceled)

30. The wound dressing of claim 1, wherein the dressing material comprises an absorbent material having a absorption capacity to absorb all fluid generated by the wound until re-epithelialization of skin on the wound.

31. The wound dressing of claim 25, wherein the dressing material comprises a biocompatible polymer, the polymer comprising a foam, a woven material, a non-woven fabric, a knitted material, or combinations thereof.

32. (canceled)

33. The wound dressing of claim 17, wherein the dressing material comprises two or more layers including at least one of an absorbent material layer and a wicking layer.

34-35. (canceled)

36. The wound dressing of claim 25, wherein the antifibrinolytic agent comprises tranexamic acid (TXA).

37. The wound dressing of claim 36, wherein the TXA has a concentration that varies across a thickness of the flexible sheet or wherein the concentration of TXA varies between layers or wherein TXA is distributed through at least a portion of the dressing material or wherein the device comprises two or more layers, and TXA is enclosed between two layers.

38-40. (canceled)

41. The wound dressing of claim 36, further comprising a backing layer with an adhesive border.

42. The wound dressing of claim 25, further comprising an antimicrobial agent in the device or applied to the dermal wound wherein the antimicrobial agent comprises a silver material or an antibiotic.

43. (canceled)

44. A tissue treatment device, comprising:
an implantable scaffold material, the scaffold material has a plurality of apertures for tissue contact through the apertures from both sides of the scaffold material; and
tranexamic acid (TXA) contained in the scaffold material for time release into a wound to reduce an exudate generated by the wound.

45. The device of claim 44, wherein the TXA is distributed within the scaffold material for timed release upon contact with fluid within the wound and the scaffold material is an extracellular matrix, a bioresorbable polymer, or combinations thereof.

46. (canceled)

47. The device of claim 44 wherein the scaffold material is sized and shaped for placement within a flap wound.

48. (canceled)

49. The device of claim 44 wherein the TXA is contained in a plurality of microspheres or porous particles.

50. A method of treating a surgical site, comprising:
performing a surgical procedure at a surgical site;
implanting a material containing tranexamic acid; and
closing the surgical site to reduce exudate within the surgical site.

51. The method of claim 50, wherein the surgical site is a breast or procedure is an abdominoplasty.

52. (canceled)

53. The method of claim 50, wherein the material comprises a tissue matrix such as an acellular dermal matrix.

54. (canceled)

55. A method of treating a skin graft donor site, comprising:
identifying a skin graft donor site;
applying a composition comprising tranexamic acid (TXA) to the skin graft donor site; and
applying a flexible liquid-impermeable covering over the skin graft donor site.

56. The method of claim 55, wherein the composition comprising TXA comprises a liquid solution applied topically to the skin graft donor site or wherein the composition comprises a wound dressing material containing TXA.

57. (canceled)

58. The method of claim 55, wherein the wound dressing material comprises a flexible sheet that includes TXA contained in at least a surface contacting region of the wound dressing material or comprises TXA at a concentration that varies across a thickness of the flexible sheet.

59-60. (canceled)

61. The method of claim 55, wherein a TXA concentration is highest at a surface contacting region and wherein the TXA concentration decreases across the thickness of the flexible sheet.

62-63. (canceled)

64. The method of claim 55, wherein the TXA is present in two or more layers having differing concentrations of TXA or wherein the TXA is configured to be released over time after placement in the site or wherein the TXA is bound to or contained within a time-release material that is biodegradable.

65-67. (canceled)

68. The method of claim 64, wherein the time release material dissolves in contact with water or wherein the time release material is a biodegradable polymer.

69. (canceled)

70. The method of claim 55, comprising applying TXA as a topical solution followed by applying the wound dressing containing TXA.
